(19) United States
(12) Patent Application Publication
(10) Pub. No.: US 2013/0052714 A1
(43) Pub. Date: Feb. 28, 2013

(54) RECOMBINANT BILIRUBIN OXIDASE AND PRODUCTION METHOD FOR THE SAME

(71) Applicants: Hideyuki Kumita, Kanagawa (JP); Daisuke Yamaguchi, Kanagawa (JP); Yuichi Tokita, Kanagawa (JP); Shuji Fujita, Tokyo (JP)

(72) Inventors: Hideyuki Kumita, Kanagawa (JP); Daisuke Yamaguchi, Kanagawa (JP); Yuichi Tokita, Kanagawa (JP); Shuji Fujita, Tokyo (JP)

(73) Assignee: SONY CORPORATION, Tokyo (JP)

(21) Appl. No.: 13/643,905

(22) PCT Filed: Apr. 25, 2011
(86) PCT No.: PCT/JP2011/060572

§ 371 Date: Oct. 26, 2012

(30) Foreign Application Priority Data
May 13, 2010 (JP) 2010-111186

(51) Int. Cl. C12N 9/02

(52) U.S. Cl. 435/189

(57) ABSTRACT

Disclosed is a method of producing a recombinant bilirubin oxidase, including a step wherein a bilirubin oxidase gene derived from imperfect filamentous fungus Myrothecium verrucaria is introduced to Escherichia coli and expression thereof is allowed. Also disclosed is a recombinant bilirubin oxidase obtained by this production method. This recombinant bilirubin oxidase has an enzymatic activity higher than that of a natural bilirubin oxidase derived from the imperfect filamentous fungus Myrothecium verrucaria.

### TECHNICAL FIELD

The present invention relates to a recombinant bilirubin oxidase and a method of producing the same. More specifically, the invention relates to a recombinant bilirubin oxidase derived from imperfect filamentous fungus myrothecium verrucaria, and the like.

### BACKGROUND ART

A “bilirubin oxidase” is an enzyme which catalyzes a reaction of oxidation of bilirubin to biliverdin, and is one kind of enzyme belonging to the multicopper oxidase (the generic term for enzymes having a plurality of copper ions in an active center thereof). Conventionally, the bilirubin oxidase has been widely used as a reagent for clinical tests of liver function and the like (a measurement reagent for bilirubin in blood serum). Besides, in recent years, the bilirubin oxidase has been used also as an electrode enzyme for catalyzing an electrochemical four-electron reduction reaction of oxygen, on the cathode (positive electrode) side of enzyme cells.

As the bilirubin oxidase, in the past, an enzyme sample obtained by extraction from imperfect filamentous fungus Myrothecium verrucaria MT-1 and partial purification of the extract (such a sample will hereinafter be referred to as “natural bilirubin oxidase”) has been used (see Patent Document 1). In addition, Non-Patent Document 1 has reported determination of the base sequence of a gene which codes a bilirubin oxidase gene, and construction of an expression system for a recombinant bilirubin oxidase by use of yeast (see Patent Document 2, as well).

### RELATED ART DOCUMENTS

### Patent Documents

Patent Document 1: Japanese Patent Laid-open No. Hei 5-199882
Patent Document 2: Japanese Patent Laid-open No. 2005-73660

### Non-Patent Document

Non-Patent Document 1: “Molecular cloning of the gene for bilirubin oxidase from Myrothecium verrucaria and its expression in yeast.” J Biol Chem. 1993 Sep. 5; 268(25): 18801-9.

### SUMMARY OF INVENTION

### Technical Problems

In order that a bilirubin oxidase derived from imperfect filamentous fungus Myrothecium verrucaria will be supplied in large amounts as a reagent for clinical tests or an electrode enzyme for enzyme cells, construction of an excellent expression system for a recombinant bilirubin oxidase has been desired. However, the conventional methods in which imperfect filamentous fungus Myrothecium verrucaria or yeast is used have had drawbacks in that the expression amount of the enzyme is small or the activity of the enzyme expressed is insufficient.

Accordingly, it is an object of the present invention to provide a simple technique for obtaining a recombinant bilirubin oxidase derived from imperfect filamentous fungus Myrothecium verrucaria with high expression and high activity.

### Technical Solution

In order to solve the above-mentioned problem, the present inventors attempted to cause a bilirubin oxidase gene derived from imperfect filamentous fungus Myrothecium verrucaria, which is a true fungus as a eukaryote, to be expressed in Escherichia coli, which is a bacterium as a prokaryote. As a result, the present inventors found out that using a simple Escherichia coli expression system, it is possible to express and purify a recombinant bilirubin oxidase having sufficient activity. Furthermore, the present inventors made clear that in an Escherichia coli expression system lacking a posttranslational sugar chain modification, unexpectedly, it is possible to obtain a recombinant bilirubin oxidase having an activity higher than that of the natural bilirubin oxidase derived from imperfect filamentous fungus Myrothecium verrucaria.

Based on these findings, according to the present invention, there is provided a recombinant bilirubin oxidase obtained by introducing a bilirubin oxidase gene derived from imperfect filamentous fungus Myrothecium verrucaria to Escherichia coli and allowing expression thereof.

This recombinant bilirubin oxidase has the amino acid sequence of SEQ ID NO: 1, and has a high enzymatic activity higher than that of the natural bilirubin oxidase derived from imperfect filamentous fungus Myrothecium verrucaria. The amino acid sequence of SEQ ID NO: 1 is a sequence obtained by removing 37 residues of a secretory signal peptide from the amino acid sequence of the natural bilirubin oxidase.

In addition, according to the present invention, there is provided a method of producing a recombinant bilirubin oxidase, including a step wherein a bilirubin oxidase gene derived from imperfect filamentous fungus Myrothecium verrucaria is introduced to Escherichia coli and expression thereof is allowed.

In the method of producing a recombinant bilirubin oxidase, it is preferable to use a bilirubin oxidase gene derived from imperfect filamentous fungus Myrothecium verrucaria which has the base sequence of SEQ ID NO: 2, and to use an Escherichia coli strain derived from K-12. The base sequence of SEQ ID NO: 2 is a sequence obtained through removal of a part which codes the secretory signal peptide.

### Advantageous Effect

According to the present invention, there is provided a simple technique for obtaining a recombinant bilirubin oxidase derived from imperfect filamentous fungus Myrothecium verrucaria with high expression and high activity.

### BRIEF DESCRIPTION OF DRAWINGS

FIG. 1 is a schematic diagram illustrating the structure of pET-BO vector.

FIG. 2 is a photograph as a substituent for a drawing, showing the results of SDS-PAGE of a purified recombinant bilirubin oxidase.

FIG. 3 is a spectrogram showing an absorption spectrum of the purified recombinant bilirubin oxidase.

FIG. 4 is a graph showing the evaluation results of activity of the recombinant bilirubin oxidase.

### MODE FOR CARRYING OUT THE INVENTION

Now, a preferred mode for carrying out the present invention will be described below referring to the drawings. Incidentally, the mode for carrying out the present invention which will be described below is an example of representative modes for carrying out the invention, and the scope of the invention is not to be thereby construed narrowly.

A recombinant bilirubin oxidase according to the present invention is obtained by introducing a bilirubin oxidase gene derived from imperfect filamentous fungus Myrothecium verrucaria to Escherichia coli and allowing expression thereof. The recombinant bilirubin oxidase is characterized by having an enzymatic activity higher than that of the natural bilirubin oxidase derived from the imperfect filamentous fungus Myrothecium verrucaria (see “Examples”). Incidentally, since the recombinant bilirubin oxidase is obtained by expression in Escherichia coli which has no posttranslational sugar chain modification, the recombinant bilirubin oxidase has no sugar chain modification, unlike the natural bilirubin oxidase and the recombinant (wild-type) bilirubin oxidase obtained by expression in yeast.

As the bilirubin oxidase gene derived from imperfect filamentous fungus Myrothecium verrucaria, a DNA fragment having the base sequence of SEQ ID NO: 2 can be used. This base sequence is said to be a sequence obtained by removal of a part coding a secretory signal peptide from a DNA which codes the natural bilirubin oxidase. The recombinant bilirubin oxidase obtained by the expression of the bilirubin oxidase gene has the amino acid sequence of SEQ ID NO: 1, which is obtained by removing 37 residues of the secretory signal peptide from the amino acid sequence of the natural bilirubin oxidase. Incidentally, the recombinant bilirubin oxidase according to the present invention may be one that has a sequence obtained by removing methionine corresponding to an initiation codon (the first methionine from the N-terminus) from the amino acid sequence of SEQ ID NO: 1.

As a vector for expression of the recombinant bilirubin oxidase, there can be adopted those plasmid vectors which are commonly used for expression of recombinant proteins. Examples of the vectors include pET22b, pUC18, pHSG398, pHSG399, pRIT2T, pUEX1 to pUEX3, pKK223-3, pINIII 1, pTTQ18, pGEMEX-1, pGH-L9, and pKK233-2.

As a host cell for expression of the recombinant bilirubin oxidase, there can be adopted those Escherichia coli cells which are commonly used for expression of recombinant proteins. As the Escherichia coli, particularly preferable is a strain derived from K-12, examples of which include such strains as BL21 (DE3), BB4, BM25.5, BMH71-18mutS, BW313, C-1a, C600, CJ236, DH1, DH5, DH5a, DH10B, DP50supF, ED8654, ED8767, ER1647, HB101, HMS174, HST02, HST04dam⁻/dcm⁻, HST08Premium, JM83, JM101, JM105, JM106, JM107, JM108, JM109, JM110, K802, K803, LE392, MC1061, MV1184, MV1193, NovaBlue, RR1, TAP90, TG1, TG2, TH2, XL1-Blue, X-1776, γ-1088, γ-1089, and γ-1090.

The introduction of the vector into the host cell (transformation) can be carried out by a conventionally known method, such as the electroporation method and the calcium method, according to the host cell to be used. In addition, extraction of the mutated bilirubin oxidase from the transformant cultured and purification of the extract can be carried out, for example, by breaking up the cells by ultrasonic pulverization, and removing unpulverized cells and precipitates from the pulverization product by centrifugation, followed by dialysis. The crude product thus obtained may be subjected further to purification by liquid chromatography.

### EXAMPLES

### Experimental Example 1

### 1. cDNA Cloning of Bilirubin Oxidase Derived from Imperfect Filamentous Fungus Myrothecium verrucaria NBRC (IFO) 6113 Strain

(1-1) Culture of Myrothecium verrucaria

Myrothecium verrucaria NBRC (IFO) 6113 strain was purchased from the independent administrative agency National Institute of Technology and Evaluation (NITE), Biotechnology Center, in lots. The purchased dried fungal body was suspended in a rehydration fluid (polypeptone: 0.50, yeast extract: 0.30, MgSO₄.7H₂O: 0.10), and the resulting suspension was inoculated into a potato dextrose agar (PDA) plate (potato dextrode: 2.40, agarose: 1.50). Upon culture at room temperature for five to seven days, the surfaces of the PDA plate were covered with white hyphae. The hyphae were scraped by a spatula, and stored at −80° C. The yield of the cell body was 50 to 60 mg (wet mass) per PDA plate (diameter: 9 cm).

(1-2) RNA Extraction and cDNA Synthesis

From about 100 mg of frozen fungal body powder of Myrothecium verrucaria NBRC (IFO) 6113 strain, 100 μm (determined by UV absorption) of total RNA was extracted. The total RNA was used as a template RNA for reverse transcription PCR.

The reverse transcription PCR was performed using the total RNA as a template and using OneStep RT-PCR Kit (produced by Qiagen K.K.). The base sequence of the primer used for the reverse transcription PCR was designed as shown in Table 1 below, referring to the base sequence of cDNA of BO which had been reported.

TABLE 1

| | | SEQ |
|---|---|---|
| Primer | Base sequence | ID No. |
| Forward | 5′- GATAT ACATA TGGTT GCCCA | 3 |
| primer | GATCA GCCCA CAGTA TC -3′ | |
| Reverse | 5′- GCTCG AATTC TTACT CGTCA | 4 |
| primer | GCTGC GGCGT AAGG -3′ | |

(In the base sequence, the underlined parts represent restriction enzyme sites, which are an NdeI site (CATATG) and an EcoRI site (GAATTC), respectively.)

As a result of agarose gel electrophoresis of the PCR product, an amplified fragment containing the desired bilirubin oxidase gene was confirmed to be present in the vicinity of 1600 bp. This amplified fragment was cut out from the agarose gel slab, and used in the subsequent step.

### (1-3) Cloning

The amplified fragment was digested by restriction enzymes NdeI and EcoRI, after which the digestion product was put to a ligation reaction with pET22b+ plasmid vector digested by the same enzymes. The plasmid vector after the ligation was introduced into Escherichia coli TOP10 strain (produced by Invitrogen Corporation), followed by inoculation onto an LB/Amp agar plate medium. After culturing overnight, a colony of a transformant having drug resistance to ampicillin was obtained. The colony was put to subculture on 3 ml of an LB/Amp medium overnight, after which a plasmid vector was isolated from the resulting cell body.

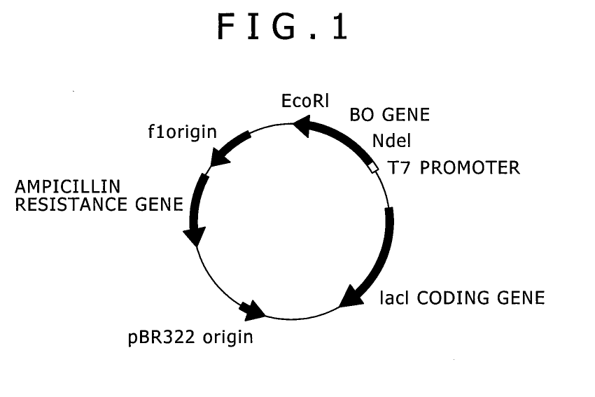

Upon examination of the base sequence of the insertion region of the plasmid vector, it was found to be the base sequence of SEQ ID NO: 2. The base sequence of SEQ ID NO: 2 is 1608 bp (inclusive of ATG as an initiation codon and TAA as a termination codon), which corresponds to 535 amino acid residues (inclusive of methionine derived from the initiation codon) described in SEQ ID NO: 1. The amino acid sequence coincided with the amino acid sequence of a mature enzyme of the natural bilirubin oxidase derived from Myrothecium verrucaria. Hereafter, this plasmid vector will be referred to as “pET-BO vector.” FIG. 1 shows the structure of the pET-BO vector.

### Experimental Example 2

### 2. Expression of Recombinant Bilirubin Oxidase in Escherichia coli

(2-1) Transformation of Escherichia coli and Expression of Recombinant Bilirubin Oxidase

Using the above-mentioned pET-BO vector, transformation of Escherichia coli (BL21 (DE3) strain derived from K-12) was carried out. The colony of the transformant thus obtained was inoculated into an LB/Amp liquid medium, and subjected to shake culture at 37° C. for four hours. Then, 1 ml of the cell body liquid thus obtained was put to subculture on 100 mL of a TB/Amp medium, and subjected to shake culture at 37° C. for four hours.

After adding IPTG (final concentration: 0.25 mM) and copper sulfate pentahydrate (2.0 mM) to the medium, culture was performed at 18° C. for 16 hours. After the culture, the resulting cell body was recovered by centrifugation. The cell body was resuspended in 50 mM of Tris-hydrochloric acid buffer (pH: 7.6, containing 0.1 mM of PMSF and 0.3 mM of copper sulfate pentahydrate), and was pulverized by ultrasonic pulverization. After unpulverized cells and precipitates were removed from the pulverization product by centrifugation, the resulting product was dialyzed with respect to 50 mM of the Tris-hydrochloric acid buffer (pH 7.6).

### (2-2) Purification of Recombinant Bilirubin Oxidase by Anion Exchange Column

Purification of the recombinant bilirubin oxidase was carried out by anion exchange chromatography based on a DEAE cellulose resin (Hi-trap DEAE (5 ml), produced by GE Healthcare). The dialyzed sample was adsorbed on the column, and the recombinant bilirubin oxidase thus adsorbed was eluted with linear gradient (0-500) using 50 mM of Tris-hydrochloric acid buffer (pH 7.6) containing 50 mM of NaCl.

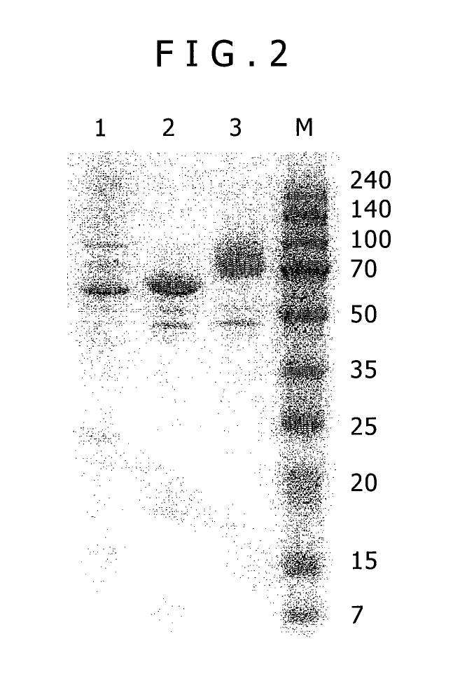

The SDS-PAGE of the recombinant bilirubin oxidase after the purification was repeated twice by the above-mentioned method is shown in FIG. 2. In the figure, lane 1 corresponds to the purified recombinant bilirubin oxidase, lane 2 corresponds to a commercially available natural bilirubin oxidase (BO) (“Amono” 3 (BO-3), produced by Amano Enzyme Inc.), and lane 3 corresponds to recombinant bilirubin oxidase obtained by expression in yeast. In addition, lane M is a size marker.

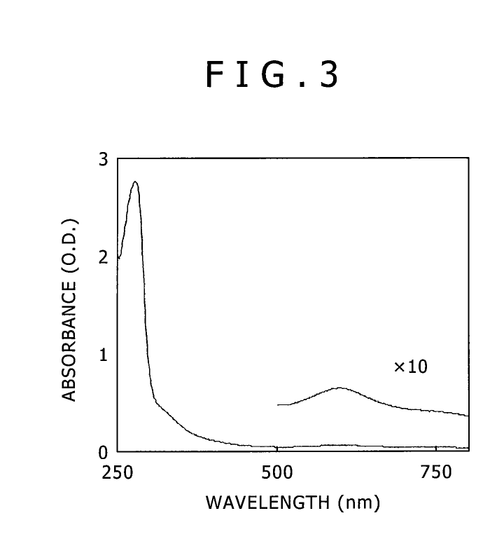

Besides, absorption spectrum of the purified recombinant bilirubin oxidase is shown in FIG. 3. The yield of the BO per 100 mL of the medium, estimated from the absorbance difference (Δε=4000) between 600 nm and 500 nm in the absorption spectrum, was 0.6 mg.

### Experimental Example 3

### 3. Measurement of Activity of Recombinant Bilirubin Oxidase

The activity of the recombinant bilirubin oxidase was evaluated, and compared with that of a commercially available natural bilirubin oxidase. The activity measurement was conducted using ABTS as a substrate, and variation in absorbance at 730 nm attendant on the progress of reaction (which arises from an increase in the amount of reaction product of ABTS) was traced. The measurement conditions are set forth in Table 2. Incidentally, the concentration of the bilirubin oxidase was so prepared that the variation in absorbance in 730 nm would be about 0.01 to 0.5 per minute. The reaction was initiated by addition of the enzyme solution (5 μL) to a phosphate buffer (2995 μL) containing 0.05 to 10 mM of ABTS.

TABLE 2

| | |
|---|---|
| Buffer | 46.5 mM aqueous Na |
| | phosphate solution (pH 7.0) |
| ABTS | 0.05 to 10 mM (final |
| concentration | concentration) |
| O₂ | saturated (210 μM; |
| concentration | 25° C.) |
| Reaction | 25° C. |
| temperature | |

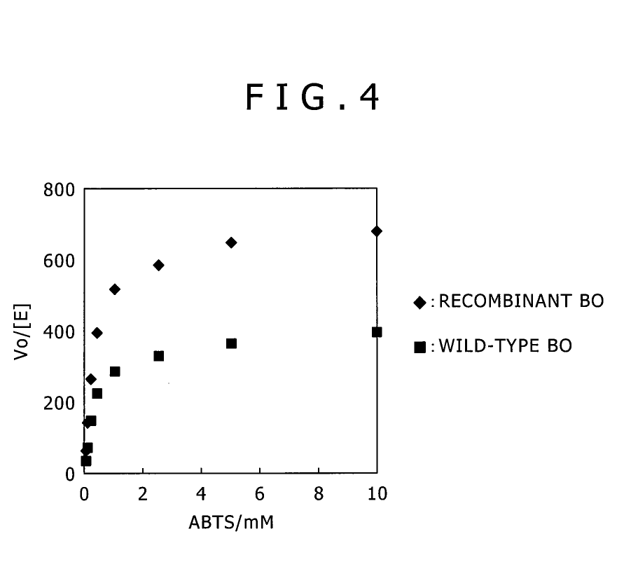

The results of the activity evaluation are shown in FIG. 4. In addition, Kₘ and k_{cat} values of the recombinant bilirubin oxidase and the commercially available natural bilirubin oxidase are set forth in Table 3. The recombinant bilirubin oxidase obtained using Escherichia coli showed an activity (k_{cat}) of about 1.8 times that of the natural bilirubin oxidase, whereby its excellent activity was verified.

TABLE 3

| | Kₘ/mM | K_{cat}/s⁻¹ |
|---|---|---|
| Recombinant BO | 0.34 ± 0.02 | 690 ± 52 |
| Wild-type BO | 0.38 ± 0.05 | 393 ± 11 |

### INDUSTRIAL APPLICABILITY

By the method of producing a recombinant bilirubin oxidase according to the present invention, a bilirubin oxidase derived from imperfect filamentous fungus Myrothecium verrucaria can be provided in large amounts through a very easy and economical method. Furthermore, the recombinant bilirubin oxidase thus obtained has a high enzymatic activity equivalent to or higher than that of the natural bilirubin oxidase. Accordingly, the present invention is applicable to inexpensive supply of a bilirubin oxidase derived from imperfect filamentous fungus Myrothecium verrucaria, as a reagent for clinical tests, an electrode enzyme for enzyme cells, or the like.

1-7. (canceled)

8. A recombinant bilirubin oxidase obtained by introducing a bilirubin oxidase gene derived from imperfect filamentous fungus Myrothecium verrucaria to Escherichia coli and allowing expression thereof, the recombinant bilirubin oxidase having an enzymatic activity higher than the enzymatic activity of a natural bilirubin oxidase derived from the imperfect filamentous fungus Myrothecium verrucaria, and the recombinant bilirubin oxidase having no sugar chain modification and having no maltose binding protein (MBP) at an N-terminus.

9. The recombinant bilirubin oxidase according to claim 8, having the amino acid sequence of SEQ ID NO: 1.

10. The recombinant bilirubin oxidase according to claim 8, used as an electrode enzyme of an enzyme cell.

11. A method of producing a recombinant bilirubin oxidase, comprising a step wherein a bilirubin oxidase gene derived from imperfect filamentous fungus Myrothecium verrucaria is introduced to Escherichia coli, and a recombinant bilirubin oxidase which has an enzymatic activity higher than the enzymatic activity of a natural bilirubin oxidase derived from imperfect filamentous fungus Myrothecium verrucaria, has no sugar chain modification, and has no maltose binding protein (MBP) at an N-terminus is expressed.

12. The method according to claim 11, wherein an Escherichia coli strain derived from K-12 is used.

13. The method according to claim 11, wherein a bilirubin oxidase gene derived from imperfect filamentous fungus Myrothecium verrucaria which has the base sequence of SEQ ID NO: 2 is used.


SEQUENCE LISTING (4 sequences)

SEQ ID NO: 1 - protein, 535 aa, Myrothecium verrucaria
MVAQISPQYPMFTVPLPIPPVKQPRLTVTNPVNGQEIWYYEVEIKPFTHQVYPDLGSADLVGYDGMSPGPTFQVPRGVETVVRFINNAEAPNSVHLHGSFSRAAFDGWAEDITEPGSFKDYYYPNRQSARTLWYHDHAMHITAENAYRGQAGLYMLTDPAEDALNLPSGYGEFDIPMILTSKQYTANGNLVTTNGELNSFWGDVIHVNGQPWPFKNVEPRKYRFRFLDAAVSRSFGLYFADTDAIDTRLPFKVIASDSGLLEHPADTSLLYISMAERYEVVFDFSDYAGKTIELRNLGGSIGGIGTDTDYDNTDKVMRFVVADDTTQPDTSVVPANLRDVPFPSPTTNTPRQFRFGRTGPTWTINGVAFADVQNRLLANVPVGTVERWELINAGNGWTHPIHIHLVDFKVISRTSGNNARTVMPYESGLKDVVWLGRRETVVVEAHYAPFPGVYMFHCHNLIHEDHDMMAAFNATVLPDYGYNATVFVDPMEELWQARPYELGEFQAQSGQFSVQAVTERIQTMAEYRPYAAADE

SEQ ID NO: 2 - DNA, 1608 bp, Myrothecium verrucaria
atggttgcccagatcagcccacagtatcccatgttcacagtaccactgccaattcctcctgttaagcagccccgcttgactgtaaccaatcctgtgaatggacaagagatctggtactatgaggtcgagatcaagcccttcactcaccaggtttaccctgaccttggatccgctgatctggtcgggtacgatggaatgtctcctggccctactttccaggttcctcgtggagttgaaacagttgtccgcttcattaacaatgctgaggctcctaactctgttcacttgcacggatcattctctcgtgccgcctttgacggatgggcagaggacatcaccgagcctggcagcttcaaagactattactacccaaacagacagtctgcccgtaccctatggtaccacgatcatgctatgcatatcactgctgagaacgcctaccgtggccaggctggtctctacatgctcactgacccagccgaagacgctctcaacttgccaagtggatatggcgagttcgatattccaatgatcctcacgtctaagcaatacaccgcaaacggcaacttggtcaccactaatggagagctgaactcattctggggtgatgtaattcacgtgaacggtcaaccctggcctttcaagaacgttgagcctcgcaaatatcgattccgcttcctcgatgccgcagtttctcgctctttcggcctttactttgctgatactgatgctatcgacactcgcttgcctttcaaggttattgcctccgattctggtcttcttgaacaccctgccgataccagcttgctgtacatttccatggccgagcgttacgaagttgtgtttgacttctccgactatgctggcaagactattgaactccgcaacctgggcggcagcattggcggcattggaacagataccgactatgataacaccgacaaggtcatgcgtttcgtggtagcagacgacacaactcagccagatacctcagttgttcctgctaaccttcgtgatgttcccttcccctctcccaccacaaacaccccccgacagttccgctttggtcgcaccggtcctacctggactattaatggtgttgcttttgctgatgttcaaaaccgtctgcttgcaaacgtacccgttggtactgtcgagcgttgggagctcatcaacgccggtaacggttggacgcaccctattcacatccatcttgtcgacttcaaggtcatttctcgtacttccggcaacaacgcgcgcacagtcatgccatacgagtccggtctcaaagacgttgtctggcttggtcgccgtgaaactgtggttgttgaggctcattacgcgcctttccctggtgtatacatgttccattgccacaatttgattcacgaggatcacgatatgatggctgcctttaacgccaccgtcctgccagattatggctataatgccactgttttcgttgaccctatggaagagctttggcaggctcgtccctatgaactcggcgagttccaggctcagagtggccagttcagcgttcaggctgttactgagcgtatccagactatggctgaatacagaccttacgccgcagctgacgagtag

SEQ ID NO: 3 - DNA, 37 bp, Artificial Sequence
  Forward pimer for RT-PCR
gatatacatatggttgcccagatcagcccacagtatc

SEQ ID NO: 4 - DNA, 34 bp, Artificial Sequence
  Reverse primer for RT-PCR
gctcgaattcttactcgtcagctgcggcgtaagg